(19) United States
(12) Patent Application Publication
(10) Pub. No.: US 2024/0366257 A1
(43) Pub. Date: Nov. 7, 2024

(54) STOMA INSTRUMENT AND STOMA METHOD

(71) Applicant: SHENZHEN BETTERWAY MEDTECH CO., LTD., Shenzhen (CN)

(72) Inventor: Lei ZHANG, Shenzhen (CN)

(73) Assignee: SHENZHEN BETTERWAY MEDTECH CO., LTD., Shenzhen, GD (CN)

(21) Appl. No.: 18/291,227

(22) PCT Filed: Apr. 29, 2022
(86) PCT No.: PCT/CN2022/090108

§ 371 Date: Jan. 23, 2024

(30) Foreign Application Priority Data
Aug. 12, 2021 (CN) 202110925953.9

(51) Int. Cl. A61B 17/3209; A61B 17/00; A61B 17/22; A61B 17/34

(52) U.S. Cl. CPC A61B 17/3209; A61B 17/22031; A61B 17/34; A61B 2017/00243

(57) ABSTRACT

A stoma instrument and a stoma method. The stoma instrument includes a first tube body, a second tube body, a third tube body, a grasping member, a puncturing member, and a cutting member. One end of the first tube body is provided with a receiving opening for receiving a biological tissue. The second tube body is movably arranged inside the first tube body. The third tube body is movably arranged inside the second tube body. The grasping member is used for radially contracting into the second tube body after deformation to follow the second tube body to pass through the biological tissue, and for radially expanding and moving, after the grasping member passes through the biological tissue and leaves the second tube body, towards the first tube body to grasp the biological tissue into the receiving opening. The cutting member is used for cutting the biological tissue at the receiving opening.

### TECHNICAL FIELD

The present application relates to the field of medical instruments, and in particular, to a stoma instrument and a stoma method.

### BACKGROUND

Heart failure is a heart disease with high incidence rate, which is divided into diastolic heart failure and systolic heart failure. For the treatment of diastolic heart failure, a small hole needs to be opened in the atrial septum of the patient's heart to form a left to right shunt, thereby reducing the pressure in the left atrium of the heart failure patient.

### Technical Problem

The stoma instruments in the existing technology generally use a balloon to expand the puncture position to form a larger opening after puncturing the atrial septal tissue. Due to the resilience of the atrial septal tissue, after the balloon is withdrawn, the tissue often contracts, leading to contracting or even closure of the opening.

### Technical Solution

In view of above, it is necessary to provide a stoma instrument and a stoma method, so that the opening is not easily contracted after the stoma.

A stoma instrument includes:
a first tube body, wherein one end of the first tube body is provided with a receiving opening for receiving a biological tissue;
a second tube body, wherein the second tube body is moveably arranged inside the first tube body;
a third tube body, wherein the third tube body is moveably arranged inside the second tube body;
a grasping member, wherein the grasping member is connected to one end of the third tube body close to the receiving opening, the grasping member is used for radially contracting into the second tube body after deformation to follow the second tube body to pass through the biological tissue, and for radially expanding and moving towards the first tube body to grasp the biological tissue into the receiving opening after the grasping member passes through the biological tissue and leaves the second tube body;
a puncturing member, wherein the puncturing member is connected to one end of the grasping member away from the third tube body, and the puncturing member is located at the end of the second tube body to guide the second tube body to pass through the biological tissue; and
a cutting member, wherein the cutting member is used for cutting the biological tissue received at the receiving opening.

In some embodiments of the stoma instrument, the grasping member includes a first connecting part connected to the third tube body, a second connecting part connected to the puncturing member, and multiple deformation parts connecting the first connecting part and the second connecting part, wherein the first connecting part and the second connecting part extend along the axial direction of the third tube body, the multiple deformation parts are arranged around the central axis of the third tube body.

In some embodiments of the stoma instrument, the deformation part is provided with a hook for grasping the biological tissue.

In some embodiments of the stoma instrument, one end of the deformation part close to the first connecting part is provided with a contacting surface for contacting the biological tissue, and the contacting surface is provided with the hook and a cutout corresponding to the hook, when the deformation part contracts, the hook is received in the cutout;
alternatively, the deformation part includes a straight section located in the middle and arc sections located and connected at two ends of the straight section, the hook is located on both sides of the straight section and extends beyond the straight section towards the first connecting part;
alternatively, the hook is V-shaped, and two ends of the hook are respectively connected to side walls of adjacent two deformation parts.

In some embodiments of the stoma instrument, the deformation part is C-shaped, and two ends of the deformation part are connected to the first connecting part and the second connecting part, respectively.

In some embodiments of the stoma instrument, the stoma instrument further includes a telescopic member that is moveably arranged inside the third tube body, and the telescopic member is fixedly connected to the second connecting part or the puncturing member to control contraction and expansion of the grasping member; and/or
the grasping member is a memory metal, when the grasping member moves out from the inner cavity of the second tube body, the grasping member returns to an expanded state.

In some embodiments of the stoma instrument, the cutting member includes a deformable cutting part disposed around the receiving opening, a first pulling part that is fixed inside the first tube body, and a second pulling part that is moveably arranged inside the first tube body, the second pulling part is used to tension the cutting part for cutting the biological tissue.

In some embodiments of the stoma instrument, the first tube body is provided with two pulling channels that penetrate axially through the first tube body, and the first pulling part and the second pulling part are respectively arranged in the two pulling channels.

In some embodiments of the stoma instrument, the cutting part is a combination of two opposite C-shaped rings.

In some embodiments of the stoma instrument, the first tube body includes an extension part and a stoma part both of which are extended axially, the center portion of the extension part is provided with an active channel for the second tube body to pass through and multiple cavities, and one end of the stoma part away from the extension part has the receiving opening, one end of the stoma part close to the extension part is provided with multiple protrusions corresponding to the cavities, and the protrusions are embedded and fixed in the cavities.

In some embodiments of the stoma instrument, the puncturing member includes a fixing part fixedly connected to the grasping member and a puncturing part for puncturing the biological tissue, and the puncturing part is hemispherical in shape.

In some embodiments of the stoma instrument, the stoma instrument further includes a control handle fixedly connected at one end of the first tube body away from the receiving opening, the second tube body and the third tube body are both connected to the control handle, and the control handle is used to control the axial movement of the second tube body and the third tube body.

A stoma method using the stoma instrument mentioned above, wherein the stoma method includes:
before stoma, the third tube body is moved, so that the grasping member is received in the second tube body, and the puncturing member is located at the end of the second tube body;
next, the second tube body is driven to move, and the grasping member and the puncturing member move with the second tube body, the puncturing member located at the end comes into contact with and punctures the biological tissue, so that the grasping member passes through the biological tissue, but the first tube body does not pass through the biological tissue;
thereafter, the second tube body is driven to move relative to the third tube body in a direction away from the puncturing member, and the grasping member deforms and expands radially after leaving the second tube body, resulting in an increase of its radial area;
then, the second tube body and the third tube body are driven to move as a whole towards the first tube body, the grasping member comes into contact with the biological tissue and pushes the biological tissue to the receiving opening; and
finally, the cutting member operates to separate the biological tissue inside the receiving opening from the biological tissue outside the receiving opening.

### Beneficial Effects

Implementing the embodiments of the present application will have the following beneficial effects:

Using the stoma instrument provided in the above embodiments, compared to the existing technology, the opening formed by cutting makes the biological tissue not prone to contracting, thereby ensuring the stability of the opening.

### BRIEF DESCRIPTION OF THE DRAWINGS

In order to more clearly illustrate the technical solutions of the embodiments of the present application, the drawings used below for the description of the embodiments will be introduced briefly. It should be understood that the following drawings only illustrate some embodiments of the present application, and therefore should not be considered as limitation to the protection scope. A person skilled in the art may further obtain other relevant drawings according to these drawings without creative work.

FIG. 1 is a structural schematic diagram of a state of the stoma instrument provided in an embodiment.

FIG. 2 is a structural schematic diagram of another state of the stoma instrument provided in FIG. 1.

FIG. 3 is a structural schematic diagram of the grasping member provided in an embodiment.

FIG. 4 is a structural schematic diagram of the grasping member provided in another embodiment.

FIG. 5 is a structural schematic diagram of the grasping member provided in a further embodiment.

FIG. 6 is a cross-sectional view of the extension part of the first tube body as shown in FIG. 1.

FIG. 7 is a structural schematic diagram of the stoma part of the first tube body as shown in FIG. 1.

FIG. 8 is a structural schematic diagram of the cutting member as shown in FIG. 1.

FIG. 9 is a structural schematic diagram of the puncturing member as shown in FIG. 1.

The reference signs in the attached drawings are as follows:
110—first tube body, 112—extension part, 1122—active channel, 1124—cavity, 114—stoma part, 1142—protrusion, 110a—receiving opening, 110b—pulling channel;
120—second tube body;
130—third tube body;
140—grasping member, 142—first connecting part, 144—second connecting part, 146 (147, 148)—deformation part, 1462 (1472, 1482)—hook, 1464—contact surface, 1474—straight section, 1476—arc section;
150—puncturing member, 152—fixing part, 154—puncturing part;
160—cutting member, 162—cutting part, 164—first pulling part, 166—second pulling part; 170—control handle.

### DETAILED DESCRIPTION OF THE EMBODIMENTS

The following will provide a clear and complete description of the technical solution in the embodiments of the present application, in conjunction with the attached drawings. Obviously, the described embodiments are only a part of the embodiments of the present application, not all of the embodiments. Based on the embodiments in the present application, all other embodiments obtained by ordinary technicians in the art without creative work fall within the scope of protection of the present application.

In the description of the embodiments of the present application, it should be noted that the terms “center”, “up”, “down”, “left”, “right”, “vertical”, “horizontal”, “inside”, “outside”, etc. indicating the orientation or position relationship are based on the orientation or position relationship shown in the attached drawings, only for convenience of describing the embodiments of the present application and simplifying the description, rather than indicating or implying that the device or component referred to must have a specific orientation or be constructed and operated in a specific orientation, therefore it cannot be understood as a limitation on the embodiments of the present application. In addition, the terms “first,” “second,” and “third” are only used for the purpose of description and cannot be understood as indicating or implying relative importance.

In the description of the embodiments of the present application, it should be noted that unless otherwise specified and limited, the terms “installed” and “connected” should be understood in a broad sense, which may be, for example, fixed connections, detachable connections, integrated connections, mechanical connections, electrical connections, direct connections, indirect connections through intermediate media, or internal connection between two components. For ordinary technicians in the art, the specific meanings of the above terms in the embodiments of the present application can be understood according to specific situations.

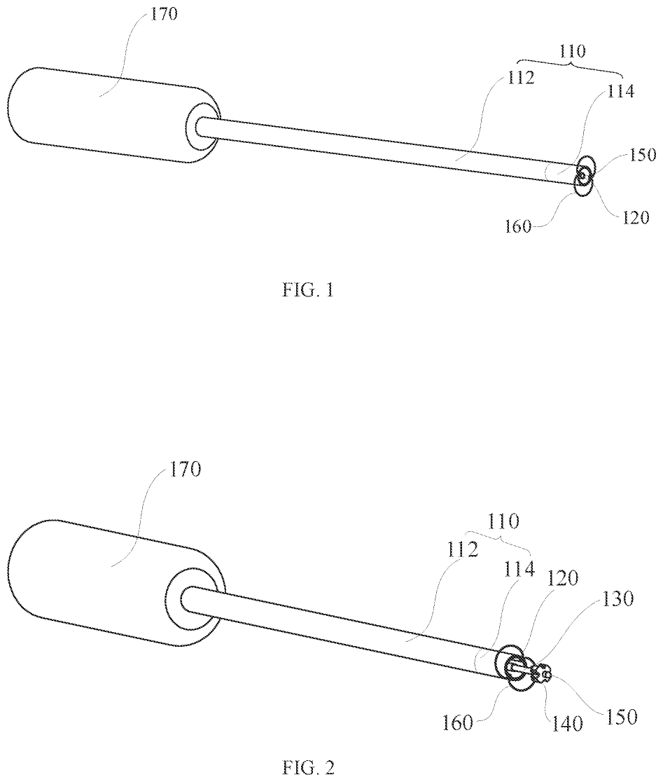

Please refer to FIGS. 1-9, an embodiment of the present application provides a stoma instrument, which includes a first tube body 110, a second tube body 120, a third tube body 130, a grasping member 140, a puncturing member 150, and a cutting member 160. One end of the first tube body 110 is provided with a receiving opening 110a for receiving a biological tissue. The second tube body 120 is moveably arranged inside the first tube body 110 along the axial direction of the first tube body 110. The third tube body 130 is moveably arranged inside the second tube body 120 along the axial direction of the second tube body 120. The grasping member 140 is connected to one end of the third tube body 130 close to the receiving opening 110a. The grasping member 140 is used for radially contracting into the second tube body 120 after deformation to follow the second tube body 120 to pass through biological tissue, and for radially expanding and moving towards the first tube body 110 to grasp the biological tissue into the receiving opening 110a after the grasping member 140 passes through the biological tissue and leaves the second tube body 120. The puncturing member 150 is connected to one end of the grasping member 140 away from the third tube body 130, and the puncturing member 150 is located at the end of the second tube body 120 to guide the second tube body 120 to pass through the biological tissue. The cutting member 160 is used for cutting the biological tissue at the receiving opening 110a.

According to the stoma instrument provided in the above embodiment, before stoma, the third tube body 130 is moved along the axial direction of the second tube body 120, so that the grasping member 140 is received in the second tube body 120, and the puncturing member 150 is located at the end of the second tube body 120. Next, the second tube body 120 is driven to move along the axial direction of the first tube body 110, the grasping member 140 and the puncturing member 150 move with the second tube body 120, and the puncturing member 150 located at the end comes into contact with and punctures the biological tissue, so that the grasping member 140 passes through the biological tissue, but the first tube body 110 does not pass through the biological tissue. Thereafter, the second tube body 120 is driven to move relative to the third tube body 130 in a direction away from the puncturing member 150 along the axial direction of the first tube body 110, and the grasping member 140 deforms and expands radially after leaving the second tube body 120, resulting in an increase of its radial area. Then, the second tube body 120 and the third tube body 130 are driven to move as a whole along the axial direction of the first tube body 110 towards the first tube body 110, the grasping member 140 comes into contact with the biological tissue and pushes the biological tissue to the receiving opening 110a. Finally, the cutting member 160 operates to separate the biological tissue inside the receiving opening 110a from the biological tissue outside the receiving opening 110a.

By using the stoma instrument provided in the above embodiment, compared to the existing technology, the opening formed by cutting makes the biological tissue not prone to contracting, thereby ensuring the stability of the opening.

It should be noted that in order to facilitate puncturing the biological tissue using the puncturing member 150, the puncturing member 150 is provided with a current circuit to puncture the biological tissue through electric cutting. However, the main protection point of the present application lies in the mechanical structure, and therefore, no specific description is here provided for the circuit connection.

In addition, the stoma instrument provided in this embodiment is applicable to but not limited to cardiac stoma surgery.

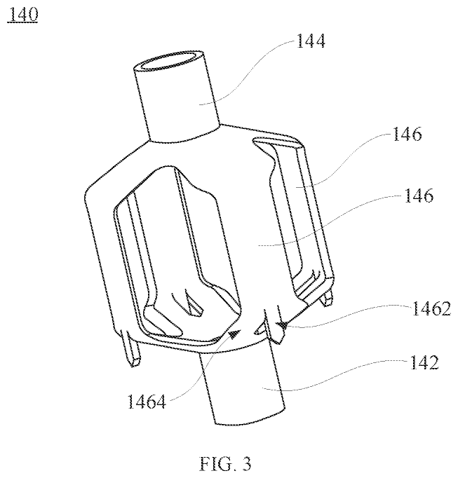

Please refer to FIG. 3, 4, or 5, in an embodiment provided in the present application, the grasping member 140 includes a first connecting part 142 connected to the third tube body 130, a second connecting part 144 connected to the puncturing member 150, and multiple deformation parts 146, 147, 148 connecting the first connecting part 142 and the second connecting part 144. The first connecting part 142 and the second connecting part 144 extend along the axial direction of the third tube body 130. The multiple deformation parts 146, 147, 148 are arranged around the central axis of the third tube body 130. The structure provided in this embodiment facilitates the radial expansion and deformation of the grasping member 140 after puncturing the biological tissue.

Optionally, the deformation parts 146, 147, 148 are C-shaped, with two ends connected to the first connecting part 142 and the second connecting part 144, respectively.

Furthermore, both the first connecting part 142 and the second connecting part 144 may be hollow cylindrical in shape. The first connecting part 142 can be sleeved with the third tube body 130, and the second connecting part 144 can be sleeved with the puncturing member 150, followed by adhesive bonding, welding, or other fixing processes.

Specifically, the puncturing member 150 includes a fixing part 152 fixedly connected to the grasping member 140 and a puncturing part 154 for puncturing the biological tissue. The puncturing part 154 is hemispherical in shape. The fixing part 152 is matched with the cylindrical second connecting part 144.

In a specific embodiment, the deformation parts 146, 147, 148 are provided with hooks for grasping the biological tissue. In this way, the phenomenon of the grasping member 140 passing through the punctured opening is avoided from occurring because of relative movement between the biological tissue and the grasping member 140.

Please refer to FIG. 3, in a more specific embodiment, one end of the deformation part 146 close to the first connecting part 142 is provided with a contact surface 1464 for contacting the biological tissue. The contact surface 1464 is provided with a hook 1462 and a cutout (not labelled) corresponding to the hook 1462. When the deformation part 146 contracts, the hook 1462 is received in the cutout. By this design, the interference of the hook 1462 during deformation is avoided, and the compactness of the structure is improved.

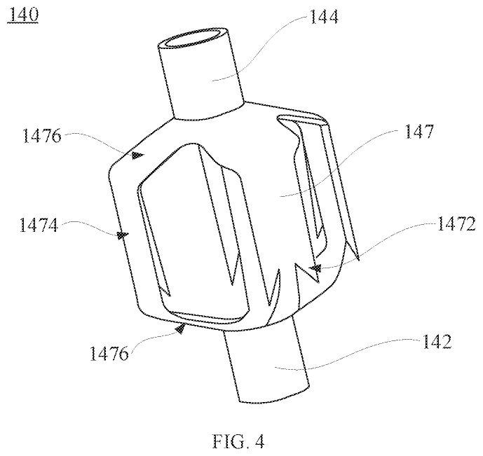

Please refer to FIG. 4, in another more specific embodiment, the deformation part 147 includes a straight section 1474 located in the middle and arc sections 1476 located and connected at two ends of the straight section 1474. The hook 1472 is located on both sides of the straight section 1474 and extends beyond the straight section 1474 towards the first connecting part 142. It can be understood that when the deformation part 147 is in an expanding state, the length of the straight section 1474 is shorter, and the hooks 1472 protrude from the straight section 1474; when the deformation part 147 is in a contracting state, the length of the straight section 1474 increases, and the hooks 1472 lie within the straight section 1474.

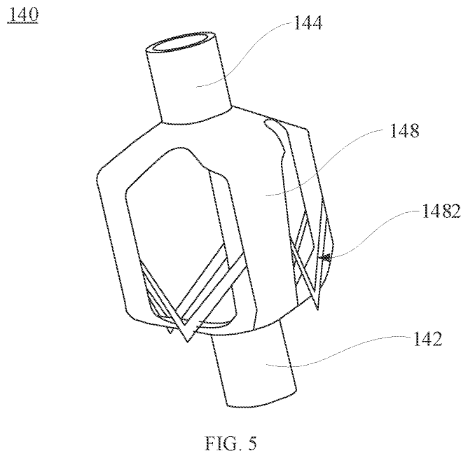

Please refer to FIG. 5, in other more specific embodiments, the hook 1482 is V-shaped, and two ends of the hook 1482 are respectively connected to side walls of adjacent two deformation parts 148. The state of the hook 1482 in this embodiment can refer to the embodiment provided in FIG. 4, and will not be specifically explained here.

In a more specific and special embodiment, the stoma instrument further includes a telescopic member (not shown) that is moveably arranged inside the third tube body 130 along the axial direction of the third tube body 130. The telescopic member is fixedly connected to the second connecting part 144 or the puncturing member 150 to control contraction and expansion of the grasping member 140. When the telescopic member moves towards the direction close to the puncturing member 150, the grasping member 140 is stretched, resulting in a contracted state, otherwise it is in an expanded state. The use of telescopic member to control the grasping member 140 can make the operation of the grasping member 140 relatively stable.

In other more specific and special embodiments, the grasping member 140 is a memory metal. When the grasping member 140 moves out from the inner cavity of the second tube body 120, the grasping member 140 returns to an expanded state. It can be understood that when the grasping member 140 is formed, the expanded state of the grasping member 140 is set as its normal state, and the grasping member 140 will automatically return to the normal state (i.e., the expanded state) when leaving the constraint of the second tube body 120. Thus, the structure is relatively simplified and the compactness of the structure is improved.

In addition, the above two methods of expanding and contracting the grasping member 140 can coexist to further ensure the stability of the grasping member 140.

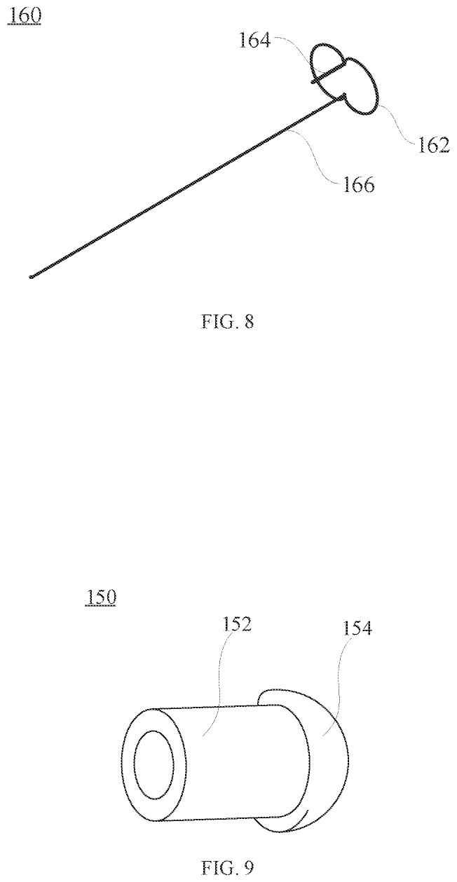

Please refer to FIG. 8, in another embodiment provided in the present application, the cutting member 160 includes a deformable cutting part 162 disposed around the receiving opening 110a, a first pulling part 164 that is fixed inside the first tube body 110, and a second pulling part 166 that is moveably arranged inside the first tube body 110 along the axial direction of the first tube body 110. The second pulling part 166 is used to tension the cutting part 162 for cutting the biological tissue. When the second pulling part 166 is pulled, the cutting part 162 is subjected to a force from the second pulling part 166, causing the annular cutting part 162 to contract and transform into a straight line from the first pulling part 164 to the second pulling part 166, while the biological tissue received in the cutting part 162 is cut by the cutting part 162 that is contracted continuously.

In another embodiment, the cutting member 160 includes a deformable cutting part 162 disposed around the receiving opening 110a, a first pulling part 164 that is moveably arranged inside the first tube body 110 along the axial direction of the first tube body 110, and a second pulling part 166 that is moveably arranged inside the first tube body 110 along the axial direction of the first tube body 110. The second pulling part 166 is used to tension the cutting part 162 in order to cut the biological tissue. When the second pulling part 166 is pulled, the cutting part 162 is subjected to a force from the second pulling part 166, causing the annular cutting part 162 to contract. Meanwhile, this force is also transmitted to the first pulling part 164 through the cutting part 162, causing the first pulling part 164 to also be pulled and move in the first tube body 110 in a direction opposite to the movement direction of the second pulling part 166. Therefore, during the cutting process, the connecting portion between the cutting part 162 and the first pulling part 164 serves as the cutting point, maintaining a state of contacting the biological tissue to be cut during the pulling process, which is conducive to achieving rapid cutting.

Of course, in order to facilitate the operation of the cutting member 160, the cutting member 160 also has a current circuit, which will not be explained in detail here.

Optionally, the cutting part 162 can be a flexible material, and before cutting, the cutting part 162 can be positioned at any position around the receiving opening 110a. The cutting part 162 can be arranged at a position corresponding to the outer diameter of the first tube body 110 or the inner diameter of the first tube body 110.

Specifically, the cutting part 162 is a combination of two opposite C-shaped rings.

In a specific embodiment, the first tube body 110 is provided with two pulling channels 110b that penetrate axially through the first tube body 110, and the first pulling part 164 and the second pulling part 166 are respectively arranged in the two pulling channels 110b.

Preferably, the connection line between the first pulling part 164 and the second pulling part 166 coincides with the diameter of the first tube body 110.

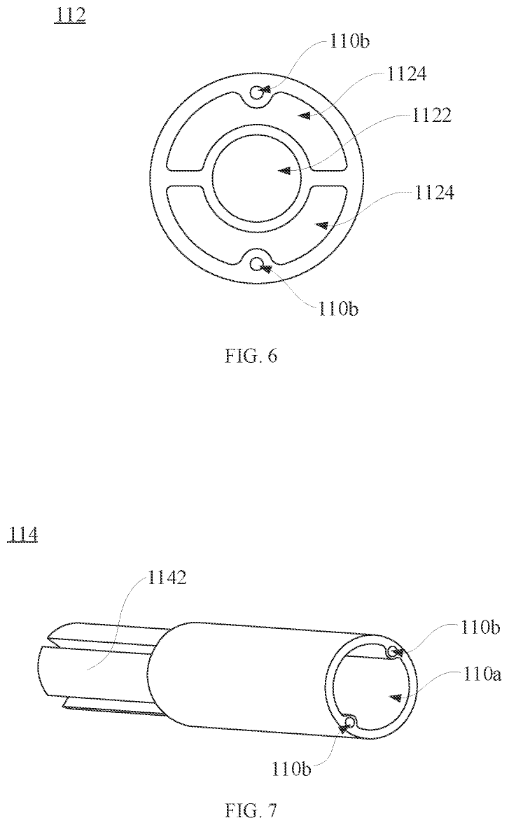

Please refer to FIGS. 6 and 7, in a more specific embodiment, the first tube body 110 includes an extension part 112 and a stoma part 114 both of which are extended axially. The center portion of the extension part 112 is provided with an active channel 1122 for the second tube body 120 to pass through and multiple cavities 1124. One end of the stoma part 114 away from the extension part 112 has the receiving opening 110a, and one end of the stoma part 114 close to the extension part 112 is provided with multiple protrusions 1142 corresponding to the cavities 1124. The protrusions 1142 are embedded and fixed in the cavities 1124. From the perspective of manufacturing, by combining the extension part 112 with the stoma part 114, the first tube body 110 with different lengths can be obtained, which is also convenient for manufacturing and reduces production and manufacturing costs.

Of course, in other more specific embodiments, the first tube body 110 can also be obtained by directly manufacturing the extension part 112.

Please refer to FIGS. 1 and 2 again, the stoma instrument further includes a control handle 170 fixedly connected at one end of the first tube body 110 away from the receiving opening 110a. The second tube body 120 and the third tube body 130 are both connected to the control handle 170, and the control handle 170 is used to control the axial movement of the second tube body 120 and the third tube body 130. However, the telescopic member and the second pulling part 166 mentioned in the above embodiments are also connected to the control handle 170. Adopting this structure can realize remote operation to avoid other factors affecting the stoma work.

The embodiment of the present application further provides a stoma method using the above stoma instrument. Specifically, the stoma method includes:

Before stoma, the third tube body 130 is moved along the axial direction of the second tube body 120, so that the grasping member 140 is received in the second tube body 120, and the puncturing member 150 is located at the end of the second tube body 120;

Next, the second tube body 120 is driven to move along the axial direction of the first tube body 110, and the grasping member 140 and the puncturing member 150 move with the second tube body 120, and the puncturing member 150 located at the end comes into contact with and punctures the biological tissue, so that the grasping member 140 passes through the biological tissue, but the first tube body 110 does not pass through the biological tissue;

Thereafter, the second tube body 120 is driven to move relative to the third tube body 130 in a direction away from the puncturing member 150 along the axial direction of the first tube body 110, and the grasping member 140 deforms and expands radially after leaving the second tube body 120, resulting in an increase of its radial area;

Then, the second tube body 120 and the third tube body 130 are driven to move as a whole along the axial direction of the first tube body 110 towards the first tube body 110, the grasping member 140 comes into contact with the biological tissue and pushes the biological tissue to the receiving opening 110a; and

Finally, the cutting member 160 operates to separate the biological tissue inside the receiving opening 110a from the biological tissue outside the receiving opening 110a.

By using the stoma method provided in the above embodiment, compared to the existing technology, the opening formed by cutting makes the biological tissue not prone to contracting, thereby ensuring the stability of the opening.

The above disclosure is only the preferred embodiments of the present application, and of course, cannot be used to limit the scope of the present application. Therefore, the equivalent changes made according to the claims of the present application still fall within the scope of the present application.

1. A stoma instrument, comprising:
a first tube body, wherein one end of the first tube body is provided with a receiving opening for receiving a biological tissue;
a second tube body, wherein the second tube body is moveably arranged inside the first tube body;
a third tube body, wherein the third tube body is moveably arranged inside the second tube body;
a grasping member, wherein the grasping member is connected to one end of the third tube body close to the receiving opening, the grasping member is used for radially contracting into the second tube body after deformation to follow the second tube body to pass through the biological tissue, and for radially expanding and moving towards the first tube body to grasp the biological tissue into the receiving opening after the grasping member passes through the biological tissue and leaves the second tube body;
a puncturing member, wherein the puncturing member is connected to one end of the grasping member away from the third tube body, and the puncturing member is located at the end of the second tube body to guide the second tube body to pass through the biological tissue; and
a cutting member, wherein the cutting member is used for cutting the biological tissue received at the receiving opening.

2. The stoma instrument as claimed in claim 1, wherein the grasping member comprises a first connecting part connected to the third tube body, a second connecting part connected to the puncturing member, and multiple deformation parts connecting the first connecting part and the second connecting part, wherein the first connecting part and the second connecting part extend along the axial direction of the third tube body, the multiple deformation parts are arranged around the central axis of the third tube body.

3. The stoma instrument as claimed in claim 2, wherein the deformation part is provided with a hook for grasping the biological tissue.

4. The stoma instrument as claimed in claim 3, wherein one end of the deformation part close to the first connecting part is provided with a contacting surface for contacting the biological tissue, and the contacting surface is provided with the hook and a cutout corresponding to the hook, when the deformation part contracts, the hook is received in the cutout;
alternatively, the deformation part comprises a straight section located in the middle and arc sections located and connected at two ends of the straight section, the hook is located on both sides of the straight section and extends beyond the straight section towards the first connecting part;
alternatively, the hook is V-shaped, and two ends of the hook are respectively connected to side walls of adjacent two deformation parts.

5. The stoma instrument as claimed in claim 2, wherein the deformation part is C-shaped, and two ends of the deformation part are connected to the first connecting part and the second connecting part, respectively.

6. The stoma instrument as claimed in claim 2, wherein the stoma instrument further comprises a telescopic member that is moveably arranged inside the third tube body, and the telescopic member is fixedly connected to the second connecting part or the puncturing member to control contraction and expansion of the grasping member; and/or
the grasping member is a memory metal, when the grasping member moves out from the inner cavity of the second tube body, the grasping member returns to an expanded state.

7. The stoma instrument as claimed in claim 1, wherein the cutting member comprises a deformable cutting part disposed around the receiving opening, a first pulling part that is fixed inside the first tube body, and a second pulling part that is moveably arranged inside the first tube body, the second pulling part is used to tension the cutting part for cutting the biological tissue;
alternatively, the cutting member comprises a deformable cutting part disposed around the receiving opening, a first pulling part that is moveably arranged inside the first tube body, and a second pulling part that is moveably arranged inside the first tube body, the second pulling part is used to tension the cutting part for cutting the biological tissue.

8. The stoma instrument as claimed in claim 7, wherein the first tube body is provided with two pulling channels that penetrate axially through the first tube body, and the first pulling part and the second pulling part are respectively arranged in the two pulling channels.

9. The stoma instrument as claimed in claim 7, wherein the cutting part is a combination of two opposite C-shaped rings.

10. The stoma instrument as claimed in claim 1, wherein the first tube body comprises an extension part and a stoma part both of which are extended axially, the center portion of the extension part is provided with an active channel for the second tube body to pass through and multiple cavities, and one end of the stoma part away from the extension part has the receiving opening, one end of the stoma part close to the extension part is provided with multiple protrusions corresponding to the cavities, and the protrusions are embedded and fixed in the cavities.

11. The stoma instrument as claimed in claim 1, wherein the puncturing member comprises a fixing part fixedly connected to the grasping member and a puncturing part for puncturing the biological tissue, and the puncturing part is hemispherical in shape.

12. The stoma instrument as claimed in claim 1, wherein the stoma instrument further comprises a control handle fixedly connected at one end of the first tube body away from the receiving opening, the second tube body and the third tube body are both connected to the control handle, and the control handle is used to control the axial movement of the second tube body and the third tube body.

13. A stoma method using the stoma instrument claimed in claim 1, wherein the stoma method comprises:
before stoma, the third tube body is moved, so that the grasping member is received in the second tube body, and the puncturing member is located at the end of the second tube body;
next, the second tube body is driven to move, and the grasping member and the puncturing member move with the second tube body, the puncturing member located at the end comes into contact with and punctures the biological tissue, so that the grasping member passes through the biological tissue, but the first tube body does not pass through the biological tissue;
thereafter, the second tube body is driven to move relative to the third tube body in a direction away from the puncturing member, and the grasping member deforms and expands radially after leaving the second tube body, resulting in an increase of its radial area;
then, the second tube body and the third tube body are driven to move as a whole towards the first tube body, the grasping member comes into contact with the biological tissue and pushes the biological tissue to the receiving opening; and
finally, the cutting member operates to separate the biological tissue inside the receiving opening from the biological tissue outside the receiving opening.

14. The stoma instrument as claimed in claim 2, wherein the cutting member comprises a deformable cutting part disposed around the receiving opening, a first pulling part that is fixed inside the first tube body, and a second pulling part that is moveably arranged inside the first tube body, the second pulling part is used to tension the cutting part for cutting the biological tissue;
alternatively, the cutting member comprises a deformable cutting part disposed around the receiving opening, a first pulling part that is moveably arranged inside the first tube body, and a second pulling part that is moveably arranged inside the first tube body, the second pulling part is used to tension the cutting part for cutting the biological tissue.

15. The stoma instrument as claimed in claim 3, wherein the cutting member comprises a deformable cutting part disposed around the receiving opening, a first pulling part that is fixed inside the first tube body, and a second pulling part that is moveably arranged inside the first tube body, the second pulling part is used to tension the cutting part for cutting the biological tissue;
alternatively, the cutting member comprises a deformable cutting part disposed around the receiving opening, a first pulling part that is moveably arranged inside the first tube body, and a second pulling part that is moveably arranged inside the first tube body, the second pulling part is used to tension the cutting part for cutting the biological tissue.

16. The stoma instrument as claimed in claim 4, wherein the cutting member comprises a deformable cutting part disposed around the receiving opening, a first pulling part that is fixed inside the first tube body, and a second pulling part that is moveably arranged inside the first tube body, the second pulling part is used to tension the cutting part for cutting the biological tissue;
alternatively, the cutting member comprises a deformable cutting part disposed around the receiving opening, a first pulling part that is moveably arranged inside the first tube body, and a second pulling part that is moveably arranged inside the first tube body, the second pulling part is used to tension the cutting part for cutting the biological tissue.

17. The stoma instrument as claimed in claim 5, wherein the cutting member comprises a deformable cutting part disposed around the receiving opening, a first pulling part that is fixed inside the first tube body, and a second pulling part that is moveably arranged inside the first tube body, the second pulling part is used to tension the cutting part for cutting the biological tissue;
alternatively, the cutting member comprises a deformable cutting part disposed around the receiving opening, a first pulling part that is moveably arranged inside the first tube body, and a second pulling part that is moveably arranged inside the first tube body, the second pulling part is used to tension the cutting part for cutting the biological tissue.

18. The stoma instrument as claimed in claim 6, wherein the cutting member comprises a deformable cutting part disposed around the receiving opening, a first pulling part that is fixed inside the first tube body, and a second pulling part that is moveably arranged inside the first tube body, the second pulling part is used to tension the cutting part for cutting the biological tissue;
alternatively, the cutting member comprises a deformable cutting part disposed around the receiving opening, a first pulling part that is moveably arranged inside the first tube body, and a second pulling part that is moveably arranged inside the first tube body, the second pulling part is used to tension the cutting part for cutting the biological tissue.
